(19) United States
(12) Patent Application Publication
(10) Pub. No.: US 2019/0192614 A1
(43) Pub. Date: Jun. 27, 2019

(54) METHOD OF PRODUCING LEUPROLIDE MICROSPHERE AND USE

(71) Applicant: Ambiopharm, Inc., North Augusta, SC (US)

(72) Inventors: Juncai Bai, North Augusta, SC (US); Simrat Singh, North Augusta, SC (US)

(21) Appl. No.: 15/886,360

(22) Filed: Feb. 1, 2018

Related U.S. Application Data
(60) Provisional application No. 62/610,465, filed on Dec. 26, 2017.

(51) Int. Cl. A61K 38/09; A61K 9/19; A61K 9/16; A61K 47/32; A61K 47/10

(52) U.S. Cl. CPC A61K 38/09; A61K 9/19; A61P 15/00; A61K 47/32; A61K 47/10; A61K 9/1694

(57) ABSTRACT

Provided herein are, inter alia, a vial comprising lyophilized leuprolide and a method of manufacturing the same.

### TECHNICAL FIELD

The present invention relates to a method of producing a leuprolide microsphere, and a vial containing the leuprolide microsphere for medical use.

### BACKGROUND

Leuprolide is a synthetic hormone used for treatment of diseases or disorders which may pertain to hormone (e.g., prostate cancer, uttering fibroids, endometriosis, and breast cancer). Leuprolide is typically administered by injection. For instance, leuprolide is administered using a prefilled dual-chamber syringe containing sterile lyophilized leuprolide, however, it also has been reported that the currently manufactured pre-filled devices cause complexity in manufacturing processes and effective dilution and delivery of the lyophilized leuprolide.

### SUMMARY OF THE INVENTION

In one aspect, provided is a method for producing a leuprolide microsphere. The method includes: preparing a mixture comprising i) leuprolide or a pharmaceutically acceptable salt thereof, ii) a first polymer, iii) a second polymer, iv) a stabilizer, v) a first solvent, and vi) a second solvent; and lyophilizing the mixture to obtain the leuprolide microsphere. The first solution may further include gelatin.

The method may further include in preparing the mixture: dissolving the first polymer in the first solvent to obtain a first solution; dissolving the second polymer and the stabilizer in the second solvent to obtain a second solution; dissolving the leuprolide or the salt thereof in the non-aqueous solvent to obtain a leuprolide solution; and combining the first solution, the second solution and the leuprolide solution.

The method may further include homogenizing the mixture.

The method may further include, before the lyophilizing the mixture, treating the mixture to evaporate the solvents to obtain a crude microsphere.

The method may further include drying, filtering and washing the crude microsphere. The method may further include resuspending the crude microsphere in a solution comprising 10% mannitol.

The leuprolide micro sphere may be formed to have the leuprolide embedded in a microsphere. The leuprolide microsphere may be formed in powder. The powder may suitably have a diameter of about 10 nm to 1 mm.

Preferably, the leuprolide may be leuprolide acetate. The first polymer may suitably include poly lactic/glycolic acid (PLGA) or poly lactic acid (PLA). The second polymer may suitably include polyvinyl alcohol (PVA). The stabilizer may suitably include mannitol. Preferably, the first solvent may suitably include dichloromethane. Preferably, the second solvent may suitably include water.

The method may further include filling the leuprolide microsphere into a container. The container may be a vial a syringe and a tube for medical use. Preferably, the leuprolide micro spheres may be Terminally Sterilized (TS).

Further provided is a vial that includes the leuprolide microsphere produced by the methods described herein. The leuprolide microsphere in the vial may be formed in a power and the leuprolide microsphere may include leuprolide embedded in a microsphere. The vial may include at least about 3.75 mg of the leuprolide embedded in the microsphere. The vial may include at least about 7.5 mg of the leuprolide embedded in the microsphere. The vial may include at least about 11.25 mg of the leuprolide embedded in the micro sphere. The vial may include at least about 30 mg of the leuprolide embedded in the microsphere. The vial may include at least about 45 mg of the leuprolide embedded in the microsphere.

Also provided is a kit including the vial as described herein. The kit may further include a diluent and a syringe. Preferably, the diluent may be mixed with the lyophilized leuprolide microsphere in the vial before use.

Other aspects of the invention are disclosed infra.

### DETAILED DESCRIPTION OF THE INVENTION

### Definition

The term “leuprolide” or “leuprorelin” as used herein refers to a compound having a structure of

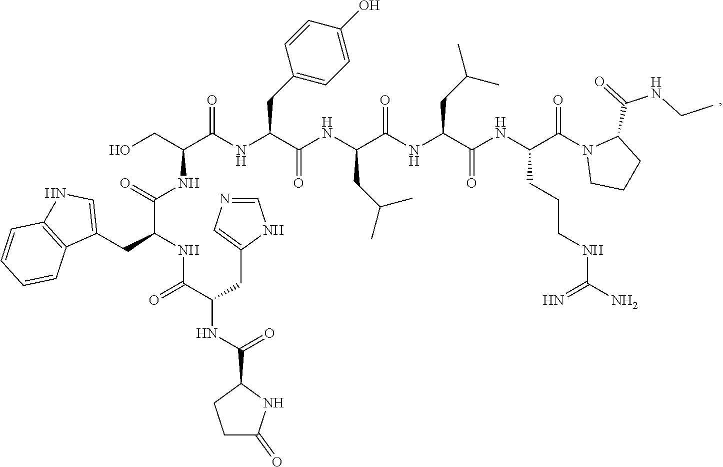 [SMILES: CCNC(=O)[C@@H]1CCCN1C(=O)[C@H](CCCNC(=N)N)NC(=O)[C@H](CC(C)C)NC(=O)[C@@H](CC(C)C)NC(=O)[C@H](Cc1ccc(O)cc1)NC(=O)[C@H](CO)NC(=O)[C@H](Cc1c[nH]c2ccccc12)NC(=O)[C@H](Cc1cnc[nH]1)NC(=O)[C@@H]1CCC(=O)N1]

or a pharmaceutically acceptable salt thereof. The leuprolide may be used for medical treatment, e.g., treating prostate cancer, breast cancer, endometriosis, uterine fibroids, and the like.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts. Preferably, the compound of the invention, leuprolide, may be provided as leuprolide acetate.

The term “polymer” as used herein refers to a molecule including repeating subunits (e.g., monomers) and created by polymerization the monomers. The polymers in the present invention may not be limited by kinds of the monomers, number or length of the polymerized monomers, arrangement of the monomers in polymer chain (e.g. linear/branched, or random copolymer/block copolymer), or molecular weight of the polymerized. Preferably, the polymers of the present invention may include at least two polymers, which may have same or different water-solubility, or otherwise same or different hydrophobicity. Examples of polymers may include, but not be limited to, naturally occurring polymers such as polynucleotides, polypeptides, proteins, and polysaccharides, and synthetic polymers such as low-density polyethylene (LDPE), high-density polyethylene (HDPE), polypropylene (PP), polyvinyl Chloride (PVC), polystyrene (PS), polyamide (e.g., Nylon, nylon 6, nylon 6,6), polytetrafluoroethylene (PTFE), polyurethanes (TPU), poly(lactic-co-glycolic acid) (PLGA), polyvinyl alcohol (PVA), polylactam, polycaprolactam, polyethylene glycol (PEG), tetraethylene glycol (TEG), polyvinylpyrrolidone (PVP), or the like. The polymers of the present invention may have a molecular weight ranges from about 1,000 Da to 1,000,000 Da, from about 2,000 Da to 1,000,000 Da, from about 3,000 Da to 1,000,000 Da, from about 4,000 Da to 1,000,000 Da, from about 5,000 Da to 1,000,000 Da, from about 10,000 Da to 1,000,000 Da, from about 10,000 Da to 500,000 Da, from about 10,000 Da to 400,000 Da, from about 10,000 Da to 300,000 Da, from about 10,000 Da to 200,000 Da, or from about 10,000 Da to 1,000,000 Da.

The term “stabilizer” or “stabilizer excipient” as used herein refers to an inactive compound or molecule that is used to prevent or reduce degradation or deformation against various conditions (e.g., light, UV-light, heat, air, pH or moisture) of other components (e.g., leuprolide) in a composition or formulation (e.g., pharmaceutical composition or medicine) during the course of manufacturing or storage after the manufacturing of the composition. The stabilizer may be polymers, sugars, salts, inorganic fillers, or the like. Preferably, the stabilizer may include sucrose, lactose, glucose, mannose, trehalose, maltose, glycerol, mannitol, sorbitol, glycine, alanine, lysine, polyethylene glycol (PEG), or dextran.

The term “microsphere” as used herein refers to a structure having a spherical shape or substantially spherical shape. Preferably, the microsphere may have a diameter or a largest diameter less than about 1000 μm (1 mm), less than about 900 μm, less than about 800 μm, less than about 700 μm, less than about 600 μm, less than about 500 μm, less than about 400 μm, less than about 300 μm, less than about 200 μm, less than about 100 μm, less than about 90 μm, less than about 80 μm, less than about 70 μm, less than about 60 μm, less than about 50 μm, less than about 40 μm, less than about 30 μm, less than about 20 μm, less than about 10 μm, less than about 1 μm, or less than about 100 nm. In embodiments, the microsphere may have a core-shell structure, which may be constituted with the same or different materials. The microsphere may be formed from an emulsion such that the core-shell structure may be formed by at least two immiscible materials. Further, the microsphere may include one or more core-shell layering structures. For instance, the microsphere may be formed to include leuprolide in the core and polymers in the shell. Alternatively, the microsphere may be formed to include one polymer in the core, leuprolide in a first shell, and the other polymer in the outer shell; however examples thereof are not limited hereto. Preferably, the leuprolide may be embedded in polymers, e.g. between layers of the polymers, between the chain of polymers, or between the mixtures of the polymers.

The term “diluent” as used herein refers to a solution or a solvent to suspend or dissolve a solid material (e.g., lyophilized powder) including active ingredient before use of the active ingredient. The diluent may substantially or completely suspend or dissolve the solid material such that the suspension may be provided as for medical use (e.g., administration or application), prior or immediately prior to the administration or application.

### Method

Provided herein is a method for producing a leuprolide microsphere powder. In one aspect, the method may produce the lyophilized leuprolide microsphere powder in a container for medical use. In another aspect, provided herein is a vial including the lyophilized leuprolide microsphere produced by the method for medical use. Further provide herein is a kit including the vial including the lyophilized leuprolide microsphere.

The method may include steps of: preparing a mixture comprising i) leuprolide or a salt thereof, ii) a first polymer, iii) a second polymer, iv) a stabilizer, v) a first solvent, and vi) a second solvent; and lyophilizing the mixture to obtain the leuprolide microsphere.

In embodiments, the first solvent and the second solvent may be immiscible. Preferably, the first solvent may be a non-aqueous solvent and the second solvent may be an aqueous solvent. The first solvent may suitably include dichloromethane. The second solvent may suitably include water.

The first polymer may include a polymer or resin component which may not be water soluble. The first polymer may include, but not limited to, poly lactic/glycolic acid (PLGA), poly lactic acid (PLA). The second polymer may include a polymer or resin component which may be water soluble or substantially water soluble. The second polymer comprises poly vinyl alcohol (PVA).

Preferably, the mixture may be prepared by dissolving the first polymer in the non-aqueous solvent to obtain a first solution; dissolving the second polymer and the stabilizer in the solvent to obtain a second solution; dissolving the leuprolide or the salt thereof in the non-aqueous solvent to obtain a leuprolide solution; and combining the first solution, the second solution and the leuprolide solution. In an exemplary embodiment, the first solution may be prepared by dissolving poly lactic/glycolic acid (PLGA) or poly lactic acid (PLA) in dichloromethane. In an exemplary embodiment, the second solution may be prepared by dissolving poly vinyl alcohol (PVA), mannitol with or without gelatin in water. In an exemplary embodiment, the leuprolide solution may be prepared by dissolving leuprolide acetate in water.

The first solution may include the first polymer in an amount of about 1 to 75 wt %, of about 1 to 70 wt %, of about 1 to 65 wt %, of about 1 to 60 wt %, of about 3 to 60 wt %, or particularly of about 5 to 50 wt % based on the total weight of the first solution. The second solution may include the second polymer in an amount of about 1 to 75 wt %, of about 1 to 70 wt %, of about 1 to 65 wt %, of about 1 to 60 wt %, of about 3 to 60 wt %, or particularly of about 5 to 50 wt % based on the total weight of the second solution. The leuprolide solution may include the leuprolide or salts thereof in an amount of about 1 to 75 wt %, of about 1 to 70 wt %, of about 1 to 65 wt %, of about 1 to 60 wt %, of about 3 to 60 wt %, or particularly of about 5 to 50 wt % based on the total weight of leuprolide first solution.

The prepared first solution, second solution and leuprolide solution may be combined without particular order. For instance, the first solution may be combined or mixed with the second solution, and the resulting mixture may be combined with the leuprolide solution. Alternatively, the first solution may be combined or mixed with the leuprolide solution, then the second solution may be added. In other exemplary embodiments, the first solution, second solution and leuprolide solution may be combined all together without specific order.

The resulting mixture, e.g. the first/second solutions, the first/leuprolide solution, or the first/second/leuprolide solutions, may be homogenized. Homogenization may be performed by using a homogenizer (e.g., ultrasonic homogenizer), shaking, vortexing, or the like, but the method thereof is not particularly limited. For example, the mixtures may be homogenized using a homogenizer, at a temperature of about 0° C. to 80° C., of about 0° C. to 70° C., of about 0° C. to 60° C., of about 0° C. to 50° C., of about 5° C. to 50° C., of about 10° C. to 50° C., of about 15° C. to 50° C., or at room temperature.

The homogenized mixture may suitably form an emulsion due to immiscible or partly miscible solvent systems (e.g. non-aqueous and aqueous solution).

Preferably, the first solution and the leuprolide solution may be combined, mixed and homogenized to form, then the second solution may be added.

The homogenized mixtures including the first solution, the second, and the leuprolide solution may be treated, e.g. heat treated, before the lyophilizing the mixture. During the treatment, the solvents in the emulsion may be substantially removed to obtain a microsphere particle. The microsphere filtrate may be optionally dried, filtered and washed. The microsphere particle may be suspended in a resuspension solution containing mannitol in an amount of about 1 wt % or greater, about 2 wt % or greater, about 3 wt % or greater, about 5 wt % or greater, about 10 wt % or greater, about 15 wt % or greater, about 20 wt % or greater, or about 25 wt % or greater, based on the total weight of the resuspension solution.

The microsphere formed in the emulsion may be lyophilized to obtain the leuprolide microsphere. Lyophilizing may be suitably performed by using a lyophilizer at a temperature less than about −10° C., less than about 20° C., ° less than about 30° C., less than about 40° C., or less than about 50° C.

Preferably, the lyophilized leuprolide microsphere may be formed in a powder. The lyophilized leuprolide microsphere may suitably have a diameter of about 10 nm to 1 mm, preferably, of about 100 nm to 500 μm, of about 100 nm to 400 μm, of about 100 nm to 300 μm, of about 100 nm to 200 μm, of about 100 nm to 100 μm, or of about 1 μm to 100 μm. In particular, in the lyophilized leuprolide microsphere, the leuprolide may be embedded in a microsphere or microstructure. The microsphere or the microstructure may be formed of the polymers, e.g. the first polymer, the second polymer, or mixtures thereof. Preferably, thus formed leuprolide microspheres may be terminally sterilized (TS).

The lyophilized leuprolide microsphere may be transferred to a container. Non-limiting examples of the container may include a vial, a syringe and a tube.

### Vial for Medical Use

Further provided herein is a vial or product thereof that includes the leuprolide microsphere produced by the methods as discussed herein. Preferably, the vial may include the leuprolide microsphere formed in a power and the leuprolide microsphere includes active compound of leuprolide embedded in microsphere or microstructure formed by the polymers (e.g., first polymer, the second polymer or combinations thereof).

In embodiments, the vial may include at least about 3.75 mg of the leuprolide embedded in the microsphere. In embodiments, the vial may include at least about 7.5 mg of the leuprolide embedded in the microsphere. In embodiments, the vial may include at least about 11.25 mg of the leuprolide embedded in the micro sphere. In embodiments, the vial may include at least about 30 mg of the leuprolide embedded in the microsphere. In embodiments, the vial may include at least about 45 mg of the leuprolide embedded in the microsphere.

### Kit

Provided herein is a kit including the vial or product thereof that includes the leuprolide microsphere produced by the methods as discussed herein. The kit may further include a diluent and a syringe. For example, by supplying the kit, the diluent may be mixed with the lyophilized leuprolide microsphere in the vial before use.

According various embodiments of the present invention, the production cost per unit may be significantly reduced for product to be presented in the vial and diluent in a syringe into a case. For instance, there are multiple facilities available globally to process the vial containing leuprolide microsphere and diluent in a syringe into a case. In addition, drying time in vial may be substantially decreased due to large surface area as compared to the front chamber of prefilled dual chamber device. Further, moisture migrating from diluent syringe into the vial may be substantially and effectively prevented in prefilled dual chamber device, which does not compromise stability of the drug product.

Moreover, according to various exemplary embodiments, vials are less complex than prefilled dual chamber device, and the manufacturing processes are fairly standard. The entire filling process and the systems with all its components (i.e. stoppers, seals, and adapters) are less complex than dial chamber systems and are therefore simpler to develop.

### Example

Provided herein below is an exemplary process of producing leuprolide microsphere powder in a vial for pharmaceutical or medical use.

PVA, mannitol and gelatin are dissolved or suspended in water to prepare W2 solution; the leuprolide acetate is dissolved or suspended in water to prepare W1 solution, and PLGA is dissolved or suspended in dichloromethane to prepare “0” solution. W1 solution and 0 solution are combined and homogenized by a homogenizer until an emulsion W1/O is formed. Thus formed W1/O emulsion is added into the W2 solution and then the resulting mixture is homogenized to obtain a double emulsion W1/O/W2. The emulsion W1/O/W2 is treated to evaporate substantial solvents (water and dichloromethane) to obtain a microsphere suspension. The microsphere suspension is filtered and washed with water to obtain a bulk microsphere particle and the bulk microsphere particle is sieved and re-suspended into 10% mannitol to produce a bulk formulated microsphere intermediate.

The bulk formulated microsphere intermediate is lyophilized by a lyophilizer to produce a bulk formulated microsphere product. The bulk formulated microsphere product is filled into vials. The vials including 3.75 mg of leuprolide microsphere, 7.5 mg of leuprolide microsphere, 11.25 mg of leuprolide microsphere, 30 mg of leuprolide microsphere, or 45 mg of leuprolide microsphere can be produced based on dosage needs. Diluting of the leuprolide microsphere may be required for reconstitution of microsphere product in the vial and the diluent may be filled separately into a glass syringe (Prefilled Syringe).

What is claimed is:

1. A method for producing a leuprolide microsphere, the method comprising:
preparing a mixture comprising i) leuprolide or a pharmaceutically acceptable salt thereof, ii) a first polymer, iii) a second polymer, iv) a stabilizer, v) a first solvent, and vi) a second solvent; and
lyophilizing the mixture to obtain the leuprolide microsphere.

2. The method of claim 1, further comprising, in preparing the mixture:
dissolving the first polymer in the first solvent to obtain a first solution;
dissolving the second polymer and the stabilizer in the second solvent to obtain a second solution;
dissolving the leuprolide or the salt thereof in the non-aqueous solvent to obtain a leuprolide solution; and
combining the first solution, the second solution and the leuprolide solution.

3. The method of claim 2, wherein the first solution further comprises gelatin.

4. The method of claim 2, further comprising homogenizing the mixture.

5. The method of claim 1, further comprising, before the lyophilizing the mixture, treating the mixture to evaporate the solvents to obtain a crude microsphere.

6. The method of claim 5, further comprising drying, filtering and washing the crude microsphere.

7. The method of claim 6, further comprising resuspending the crude microsphere in a solution comprising an amount of about 10 wt % of mannitol based on the total weight of the solution.

8. The method of claim 1, wherein the leuprolide is leuprolide acetate.

9. The method of claim 1, wherein the leuprolide microsphere is formed to have the leuprolide embedded in a microsphere.

10. The method of claim 1, wherein the leuprolide microsphere is formed in powder.

11. The method of claim 10, wherein the powder has a diameter of about 10 nm to 1 mm.

12. The method of claim 1, wherein the first polymer comprises poly lactic/glycolic acid (PLGA) or poly lactic acid (PLA).

13. The method of claim 1, wherein the second polymer comprises polyvinyl alcohol (PVA).

14. The method of claim 1, wherein the stabilizer comprises mannitol.

15. The method of claim 1, wherein the first solvent comprises dichloromethane.

16. The method of claim 1, wherein the second solution comprises water.

17. The method of claim 1, further comprising filling the leuprolide microsphere into a container.

18. The method of claim 18, wherein the container is a vial a syringe for medical use.

19. The method of claim 1, wherein the leuprolide microspheres is terminally sterilized.

20. A vial comprising a leuprolide microsphere produced by a method of claim 1.

21. The vial of claim 20, wherein the leuprolide microsphere is formed in a power.

22. The vial of claim 20, wherein the leuprolide microsphere comprises leuprolide embedded in a microsphere.

23. The vial of claim 22, wherein the vial comprises at least about 3.75 mg of the leuprolide embedded in the microsphere.

24. The vial of claim 22, wherein the vial comprises at least about 7.5 mg of the leuprolide embedded in the microsphere.

25. The vial of claim 22, wherein the vial comprises at least about 11.25 mg of the leuprolide embedded in the microsphere.

26. The vial of claim 22, wherein the vial comprises at least about 30 mg of the leuprolide embedded in the microsphere.

27. The vial of claim 23, wherein the vial comprises at least about 45 mg of the leuprolide embedded in the microsphere.

28. A kit comprising a vial of claim 20.

29. The kit of claim 28, further comprising a diluent and a syringe.

30. The kit of claim 29, wherein the diluent is mixed with the lyophilized leuprolide microsphere in the vial before use.
